(19) United States
(12) Patent Application Publication
(10) Pub. No.: US 2012/0136386 A1
(43) Pub. Date: May 31, 2012

(54) Heat-Generating Device, Heat-Generating Method and Biological Tissue-Bonding Device

(76) Inventors: Akio Kishida, Koto-ku (JP); Tsuyoshi Kimura, Koto-ku (JP); Seiichi Funamoto, Sapporo-shi (JP); Toru Masuzawa, Hitachi-shi (JP)

(21) Appl. No.: 13/375,732

(22) PCT Filed: Jun. 2, 2010
(86) PCT No.: PCT/JP2010/059375

§ 371 Date: Jan. 5, 2012

(30) Foreign Application Priority Data
Jun. 3, 2009 (JP) 2009-133992

(51) Int. Cl. A61B 17/03; D06F 75/08

(52) U.S. Cl. 606/213; 219/245

(57) ABSTRACT

The present invention provides: a heat-generating device which includes a heat-generating unit 5a, having a resin member 10a which generates heat upon application of vibration and a vibration unit 11a which imparts vibration to the resin member 10a, and a heat generation control unit 6a which, by controlling the vibration generated by the vibration unit 11a, controls the heat generation of the heat-generating unit 5a in such a manner that the heat-generating unit 5a has a prescribed temperature; a heat-generating method utilizing this device; and a biological tissue-bonding device 1a utilizing the device.

### TECHNICAL FIELD

The present invention relates to a heat-generating device, heat-generating method and a biological tissue-bonding device.

### BACKGROUND ART

Conventionally, in order to bond biological tissues together, surgical suture threads, adhesives, automatic anastomotic devices, staplers, clips and the like have been used. However, surgical suture threads have problems in that, for example, suturing is time-consuming (especially surturing micro parts) and requires skills, and adhesives (such as fibrin pastes and cyanoacrylates) have problems in their low bonding strength, low safety (for instance, fibrin pastes may cause infection and cyanoacrylates may cause cancer) and the like. Further, automatic anastomotic devices have problems in that, for example, application thereof to a micro site is difficult, and staplers, clips and the like are problematic in that, for example, a long time is required for bonding.

Meanwhile, although biological tissues can be coagulated and bonded together using an ultrasonic scalpel (vibration mode), it is difficult to make the device compact since it requires a horn for obtaining a large vibration amplitude. It is believed that biological tissues are bonded with an ultrasonic scalpel as a result of partial fusion of the collagen matrices of the biological tissues by friction heat generated by ultrasonic vibration of the scalpel blade. A high-frequency scalpel can bond biological tissues with heat (approximately 100° C.) generated by high frequency vibration; however, its large scalpel portion damages the periphery portion. An electrocautery scalpel (hemostasis mode) can stop hemorrhage by burning off biological tissues at high temperature (approximately 300° C.); however, it is difficult to bond biological tissues with an electrocautery scalpel.

There are disclosed inventions whose object is to provide a device for bonding a biological tissue with another biological tissue, or with a material capable of being bonded to a biological tissue (for example, see Japanese Patent Application Laid-Open (JP-A) No. 2007-229270).

### PRIOR ART DOCUMENT

### Patent Document

[Patent Document 1] JP-A No. 2007-229270

### SUMMARY OF THE INVENTION

### Technical Problem to be Addressed by the Invention

An object of the present invention is to provide a novel heat-generating device and a novel heat-generating method, as well as a novel biological tissue-bonding device utilizing the heat-generating device.

### Solution to Problem

In order to solve the problems, the heat-generating device according to the present invention includes a heat-generating unit having a resin member which generates heat upon application of vibration and a vibration part which imparts vibration to the resin member; and a heat generation control unit which, by controlling the vibration applied by the vibration part, controls the heat generation of the heat-generating unit in such a manner that the heat-generating unit has a prescribed temperature.

In the heat-generating device according to the present invention, by applying vibration to the resin member in the heat-generating unit, a portion of the resin member to which vibration has been applied generates heat.

In the heat-generating device according to the present invention, by controlling the vibration applied to the resin member from the vibration part by the heat generation control unit, heat generation of the heat-generating unit is controlled in such a manner that the heat-generating unit has a prescribed temperature.

In the heat-generating device according to the present invention, since a portion of the resin member to which vibration has been applied generates heat, the range for generating heat of the resin member can be determined by adjusting the range for applying vibration.

Further, the heat-generating device according to the present invention is different from conventional heat-generating devices, such as electric heaters, in that it is necessary to supply an electric current to the resin member in order to allow the heat-generating unit to generate heat. Therefore, the heat-generating device according to the present invention can be used for heating a member being susceptible to an electric field.

The direction of the vibration to be applied to the resin member may be either parallel or perpendicular to a surface of the resin member that contacts the vibration part; however, in order to apply the vibration energy efficiently, the direction of the vibration is preferably perpendicular to the surface of the resin member that contacts the vibration part.

In the heat-generating device according to the present invention, the prescribed temperature is preferably lower than the melting point of the resin member, or lower than 250° C. In that case, deformation or destruction of the resin member due to heat, or breakage etc. of the vibration part due to heat, can be prevented.

The resin member which can be used in the heat-generating device according to the present invention is preferably at least one selected from the group consisting of polytetrafluoroethylene, polyvinylidene fluoride, polyvinyl fluoride, perfluoroalkoxy fluorine resin, tetrafluoroethylene/hexafluoropropylene copolymer, ethylene/tetrafluoroethylene copolymer and ethylene/chlorotrifluoroethylene copolymer is preferred. The resin member is preferred because of its high heat generation performance upon application of vibration, and excellent heat resistance.

The first biological tissue-bonding device according to the present invention is a biological tissue-bonding device for bonding a biological tissue, which is a first adherend, with another biological tissue or a material capable of being bonded to a biological tissue, which is a second adherend, the device having a clamping part which clamps the first and second adherends so as to contact each other; a clamping force control unit which controls the clamping force exerted by the clamping part in such a manner that a pressure of 9×10² to 1×10⁵ N/m² is applied to the first and second adherends being clamped by the clamping part; a heat-generating unit which heats at least one of the first or second adherends, the heat-generating unit including a resin member which generates heat upon application of vibration and a vibration part which imparts vibration to the resin member; a heat generation control unit which, by controlling the vibration applied by the vibration part, controls the heat generation of the heat-generating unit in such a manner that the first and second adherends clamped by the clamping part have a temperature of 60 to 140° C.; a vibration unit which vibrates at least one of the first or second adherends clamped by the clamping part; and a vibration control unit which controls the vibration applied by the vibration unit in such a manner that the first and second adherends clamped by the clamping part vibrate at a frequency of 1 to 100 kHz.

In the first biological tissue-bonding device according to the present invention, the clamping part clamps the first and second adherends being in contact with each other.

In the first biological tissue-bonding device according to the present invention, the clamping force exerted by the clamping part is controlled by the clamping force control unit, so that a pressure of 9×10² to 1×10⁵ N/m² is applied to the first and second adherends being clamped by the clamping part.

In the first biological tissue-bonding device according to the present invention, heat generation of the heat-generating unit is controlled by the heat generation control unit, so that the first and second adherends being clamped by the clamping part are heated to a temperature of 60 to 140° C. The heat-generation unit heats at least one of the first or second adherends. However, since the first and second adherends are in contact with each other, even if only one of them is heated, the heat applied thereto is transmitted to the other adherend to heat the same.

In the first biological tissue-bonding device according to the present invention, the vibration generated by the vibration unit is controlled by the vibration control unit, and the first and second adherends being clamped by the clamping part vibrate at a frequency of 1 to 100 kHz. The vibration unit vibrates at least one of the first or second adherends. However, since the first and second adherends are in contact with each other, even if only one of them is vibrated, the vibration applied thereto is transmitted to the other adherend to vibrate the same. The direction of the vibration applied to the first and second adherends is not particularly restricted and, for example, it may be substantially parallel to the contact surface of the first and second adherends, or it may be substantially perpendicular to the contact surface of the first and second adherends.

Accordingly, in the first biological tissue-bonding device according to the present invention, the first and second adherends being clamped by the clamping part are in contact with each other and subjected to a pressure of 9×10² to 1×10⁵ N/m², a temperature of 60 to 140° C. and a vibration having a frequency of 1 to 100 kHz. As a result, the first and second adherends are bonded quickly and strongly. Furthermore, when the pressure, the temperature and the vibration as mentioned above are applied to the first and second adherends, the damage to the adherends is suppressed. The pressure, temperature and frequency of vibration that are applied to the first and second adherends are preferably 1×10⁴ to 5×10⁴ N/m², 80 to 110° C. and 10 to 60 kHz, respectively.

In the first biological tissue-bonding device according to the present invention, it is preferred that the vibration control unit control the vibration generated by the vibration unit in such a manner that the first and second adherends being clamped by the clamping part vibrate at an amplitude of less than 100 μm.

The amplitude of the vibration applied to the first and second adherends is not particularly restricted, as long as the first and second adherends are subjected to a pressure of 9×10² to 1×10⁵ N/m², a temperature of 60 to 140° C. and a vibration at a frequency of 1 to 100 kHz. However, in order to attain an amplitude of not smaller than 100 μm, it is difficult to reduce the size of the device due to the need for providing a large vibration element, a horn or the like. In contrast to this, in the first biological tissue-bonding device according to the present invention, when the first and second adherends clamped by the clamping part are vibrated at an amplitude of less than 100 μm, since a compact vibration element can be used and there is no need for a horn, the size of the device can be reduced. By reducing the size of the device, it becomes possible to utilize the same in endoscopic surgeries, endovascular treatments and the like.

The constitution of the first biological tissue-bonding device according to the present invention can be appropriately modified in accordance with, for example, the thickness of the first and second adherends. Here, the thicknesses of the first and second adherends refer to thicknesses in a direction perpendicular to the contact surface of the first and second adherends.

The first biological tissue-bonding device according to the present invention may have a constitution in which, for example, the heat-generating unit contacts one of the first and second adherends, which are clamped by the clamping part and are in contact with each other, the heat-generating unit heating the adherend being in contact with the heat-generating unit, and the vibration unit vibrates at least one of the clamping part or the heat-generating unit, thereby vibrating at least one of the first or second adherends. In that case, in order to make it easier to apply a pressure of 9×10² to 1×10⁵ N/m², a temperature of 60 to 140° C. and a vibration at a frequency of 1 to 100 kHz to portions of the first and second adherends at which the first and second adherends are bonded to each other, the thicknesses of the first and second adherends are preferably small, and are usually 0.01 to 5 mm, preferably 0.1 to 1 mm.

The first biological tissue-bonding device according to the present invention may have a constitution in which, for example, the heat-generating unit is interposed between the first and second adherends that are clamped by the clamping part and are in contact with each other, the heat-generating unit heating at least one of the first or second adherends that are clamped by the clamping part, and the vibration unit vibrates the heat-generating unit, thereby vibrating at least one of the first or second adherends that are clamped by the clamping part. In that case, since it is easy to apply a pressure of 9×10² to 1×10⁵ N/m², a temperature of 60 to 140° C. and a vibration at a frequency of 1 to 100 kHz to portions of the first and second adherends at which the first and second adherends are bonded to each other, the thicknesses of the first and second adherends may be large, and are usually 0.01 to 10 mm, preferably 0.1 to 5 mm.

The first biological tissue-bonding device according to the present invention may have a constitution in which, for example, the heat-generating unit and the heat generation control unit also serve as the vibration unit and the vibration control unit, and the heat-generating unit vibrates at least one of the first or second adherends that are clamped by the clamping part. In the heat-generating unit, vibration is applied to the resin member by the vibration part, and allows at least one of the first or second adherends to vibrate. By controlling the vibration of the vibration part at a frequency of 1 to 100 kHz, preferably 10 to 60 kHz using the heat-generation controlling unit, a prescribed vibration is applied to at least one of the first or second adherends from the heat-generating unit. By using the heat-generating unit and the heat generation control unit which also serve as the vibration unit and the vibration control unit, it becomes possible to further reduce the size of the biological tissue-bonding device.

The second biological tissue-bonding device according to the present invention is a biological tissue-bonding device for bonding a biological tissue, which is a first adherend, and a biological tissue or a material capable of being bonded to a biological tissue, which is a second adherend. The device has a pressing part which presses one of the first or second adherends against the other adherend; a pressure control unit which controls the pressure exerted by the pressing part in such a manner that a pressure of 9×10² to 1×10⁵ N/m² is applied to the first and second adherends; a heat-generating unit having a resin member which generates heat upon application of vibration and a vibration part which imparts vibration to the resin member, the heat-generating unit heating at least one of the first or second adherends; a heat generation control unit which controls the vibration applied by the vibration part, thereby controlling the heat generation of the heat-generating unit in such a manner that the first and second adherends, being pressed by the pressing part, have a temperature of 60 to 140° C.; a vibration unit which vibrates at least one of the first or second adherends; and a vibration control unit which controls the vibration generated by the vibration unit in such a manner that the first or second adherends vibrate at a frequency of 1 to 100 kHz.

In the second biological tissue-bonding device according to the present invention, the pressing part presses one of the first or second adherends against the other adherend, thereby allowing them to contact each other.

In the second biological tissue-bonding device according to the present invention, the pressure exerted by the pressing part is controlled by the pressure control unit, and a pressure of 9×10² to 1×10⁵ N/m² is applied to the first and second adherends. In order to apply a pressure to the first and second adherends, it is necessary that one of the first or second adherends pushes back the other adherend as a counteraction of being pressed against the other adherend. Therefore, the adherend which is to be pressed is selected from an adherend capable of counteraction (for example, a tissue being fixed to a living body, such as a blood vessel).

In the second biological tissue-bonding device according to the present invention, the heat generation of the heat-generating unit is controlled by the heat generation control unit, and the first and second adherends are heated to a temperature of 60 to 140° C. The heat-generation unit heats one or both of the first and second adherends. Since the first and second adherends are in contact with each other, even if only one of them is heated, the heat applied thereto is transmitted to the other adherend to heat the same.

In the second biological tissue-bonding device according to the present invention, the vibration generated by the vibration unit is controlled by the vibration control unit, and the first and second adherends vibrate at a frequency of 1 to 100 kHz. The vibration unit vibrates one or both of the first and second adherends. However, since the first and second adherends are in contact with each other, even if only one of them is vibrated, the vibration applied thereto is transmitted to the other adherend to vibrate the same. Further, the direction of the vibration applied to the first and second adherends is not particularly restricted and, for example, it may be substantially parallel to the contact surface of the first and second adherends, or it may be substantially perpendicular to the contact surface of the first and second adherends.

Accordingly, in the second biological tissue-bonding device according to the present invention, the first and second adherends being clamped by the clamping part are in contact with each other and subjected to a pressure of 9×10² to 1×10⁵ N/m², a temperature of 60 to 140° C. and a vibration at a frequency of 1 to 100 kHz. In that case, the first and second adherends are bonded quickly and strongly. Furthermore, when the pressure, the temperature and the vibration as mentioned above are applied to the first and second adherends, the damage to the first and second adherends is suppressed. The pressure, the temperature and the frequency of the vibration to be applied to the first and second adherends are preferably 1×10⁴ to 5×10⁴ N/m², 80 to 110° C. and 10 to 60 kHz, respectively.

In the second biological tissue-bonding device according to the present invention, it is preferred that the vibration control unit control the vibration generated by the vibration unit in such a manner that the first and second adherends vibrate at an amplitude of less than 100 μm.

The amplitude of the vibration applied to the first and second adherends is not particularly restricted, as long as the first and second adherends are subjected to a pressure of 9×10² to 1×10⁵ N/m², a temperature of 60 to 140° C. and a vibration at a frequency of 1 to 100 kHz. In the second biological tissue-bonding device according to the present invention, when the first and second adherends are vibrated at an amplitude of less than 100 μm, since a compact vibration element can be used and there is no need to provide a horn, the size of the device can be reduced. By reducing the size of the device, it becomes possible to utilize the same in endoscopic surgeries, endovascular treatments and the like.

The second biological tissue-bonding device according to the present invention may have a constitution in which, for example, the heat-generating unit contacts one of the first or second adherends, which are pressed by the pressing part and are in contact with each other; the heat-generating unit heating the adherend being in contact with the heat-generating unit, and the vibration unit vibrates the adherend being in contact with the heat-generating unit by vibrating the heat-generating unit. In this constitution, in order to ensure the contact between the first or second adherend and the heat-generating unit, it is preferred that the pressing part press the heat-generating unit against one of the first or second adherends, so that the first and second adherends are pressed against each other. By pressing the heat-generating unit against one of the first or second adherends by the pressing part, contact of the heat-generating unit with one of the first or second adherends can be ensured.

The second biological tissue-bonding device according to the present invention may have a constitution in which, for example, the heat-generating unit and the heat generation control unit also serve as the vibration unit and the vibration control unit such that the heat-generating unit vibrates the adherend being in contact with the heat-generating unit. In the heat-generating unit, vibration is applied to the resin member by the vibration part, and allows the adherend being in contact with the heat-generating unit to vibrate. By controlling the vibration of the vibration part to a frequency of 1 to 100 kHz, preferably 10 to 60 kHz, with the heat-generation controlling unit, a prescribed vibration is applied to the adherend being in contact with the heat-generating unit from the heat-generating unit. By using the heat-generating unit and the heat generation control unit which also serve as the vibration unit and the vibration control unit, it becomes possible to further reduce the size of the biological tissue-bonding device.

In the heat-generating method according to the present invention, the resin member is allowed to heat by applying vibration to the resin member that generates heat upon application of vibration.

In the heat-generating method according to the present invention, since the resin member generates heat only at a portion to which vibration has been applied, the range of the resin member at which heat is generated can be determined in advance by adjusting the range to which vibration is to be applied.

The direction of the vibration to be applied to the resin member may be parallel or perpendicular to a surface of the resin member that contacts the vibration part. However, in order to apply vibration energy efficiently, the direction of vibration is preferably perpendicular to the surface of the resin member that contacts the vibration part. The resin member suitable for the heat-generating method according to the present invention is the same as the resin member of the heat-generating device according to the present invention, as described above.

### Effects of the Invention

According to the present invention, a novel heat-generating device and a novel heat-generating method, as well as a novel biological tissue-bonding device utilizing the heat-generating device, are provided.

### BRIEF DESCRIPTION OF DRAWINGS

FIG. 1 is a partial cross-sectional schematic diagram showing the biological tissue-bonding device according to a first embodiment of the invention.

FIG. 2 is a partial cross-sectional schematic diagram showing the biological tissue-bonding device according to a second embodiment of the invention.

FIG. 3 is a partial cross-sectional schematic diagram showing the biological tissue-bonding device according to a third embodiment of the invention.

FIG. 4 is a graph showing the evaluation results of the samples 1 and 2 in the Examples.

FIG. 5 is a graph showing the evaluation results of the samples 1 and 3 in the Examples.

### DESCRIPTION OF EMBODIMENTS

The present invention will now be described in detail with reference to the drawings.

### First Embodiment

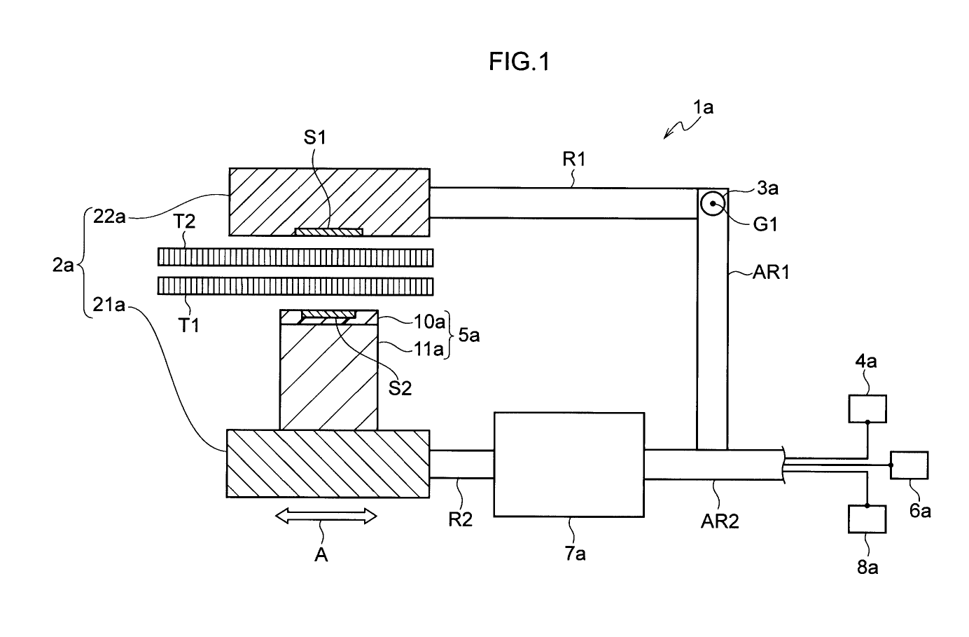

The biological tissue-bonding device 1a according the first embodiment is a biological tissue-bonding device for bonding adherends T1 and T2. The device has, as shown in FIG. 1, a heat-generating unit 5a including a resin member 10a which generates heat upon application of vibration and a vibration part 11a which imparts vibration to the resin member 10a, such that the resin member 10a contacts the adherend T1; a clamping part 2a which clamps the adherends T1 and T2 being positioned between a member 21a which is configured integrally with the heat-generating unit 5a and a member 22a; a pressing part 3a which presses the member 22a in a direction toward the member 21a; a clamping force control unit 4a which controls the pressure (clamping force) exerted by the pressing part 3a; a heat generation control unit 6a which, by controlling the vibration of the vibration part 11a, controls heat generation of the heat-generating unit 5a in such a manner the heat-generating unit 5a has a prescribed temperature; a vibration unit 7a which generates microvibration; and a vibration control unit 8a which controls the microvibration generated by the vibration unit 7a.

The types of the adherends T1 and T2 are not particularly restricted. Both of the adherends T1 and T2 may be a biological tissue, or either one of them may be a biological tissue with the other being a material capable of being bonded to a biological tissue. Examples of the biological tissue include cardiovascular tissue, gastrointestinal tissue, dermal tissue, tendon tissue, ligament tissue, mesenchymal/parenchymal tissue, vascular tissue, metabolic tissue, brain tissue, lymphoid tissue and muscle tissue. The material capable of being bonded to a biological tissue is not particularly restricted as long as it can be bonded to a biological tissue when it is subjected to a pressure of 9×10² to 1×10⁵ N/m² (preferably 1×10⁴ to 5×10⁴N/m²), a temperature of 60 to 140° C. (preferably 80 to 110° C.) and a vibration at a frequency of 1 to 100 kHz (preferably 10 to 60 kHz), and examples of the material include wet collagen, polyurethane, vinylon, gelatin and composite materials thereof. The adherends T1 and T2 may be made of a biological tissue-bonding material by itself, or may be a medical instrument having a portion made of a material capable of being bonded to a biological tissue. Examples of the medical instrument include a stent, a stent-graft (covered stent) an artificial blood vessel, an adhesion-preventing film, a wound-dressing material, a vascular catheter, a cannula, a monitoring tube, an artificial kidney, an artificial heart-lung apparatus, a blood circuit for extracorporeal circulation, an A-V shunt for an artificial kidney, an artificial blood vessel, an artificial heart, a prosthetic cardiac valve, a temporary blood bypass tube, a blood circuit for dialysis, a blood bag, a disposable circuit for apheresis system, a dialysis membrane, an artificial liver, a nanoparticle cover material, a biosensor covering material, a percutaneous device, an arteriovenous shunt, a cardiac pacemaker, an intravenous hyperalimentation catheter and a heart-wrapping net. The thicknesses of the adherends T1 and T2 (the thicknesses in a direction perpendicular to the contact surface of the adherends T1 and T2) are not particularly restricted, but are usually 0.01 to 5 mm, preferably 0.1 to 1 mm. The device 1a according to the first embodiment is suitable for bonding adherends having a relatively small thickness.

In a case where the adherends T1 and T2 are a combination of a biological tissue and a material capable of being bonded to a biological tissue, the bonding strength between the biological tissue and the material capable of being bonded to a biological tissue is usually 0.1 to 2 MPa, preferably 0.5 to 1 MPa.

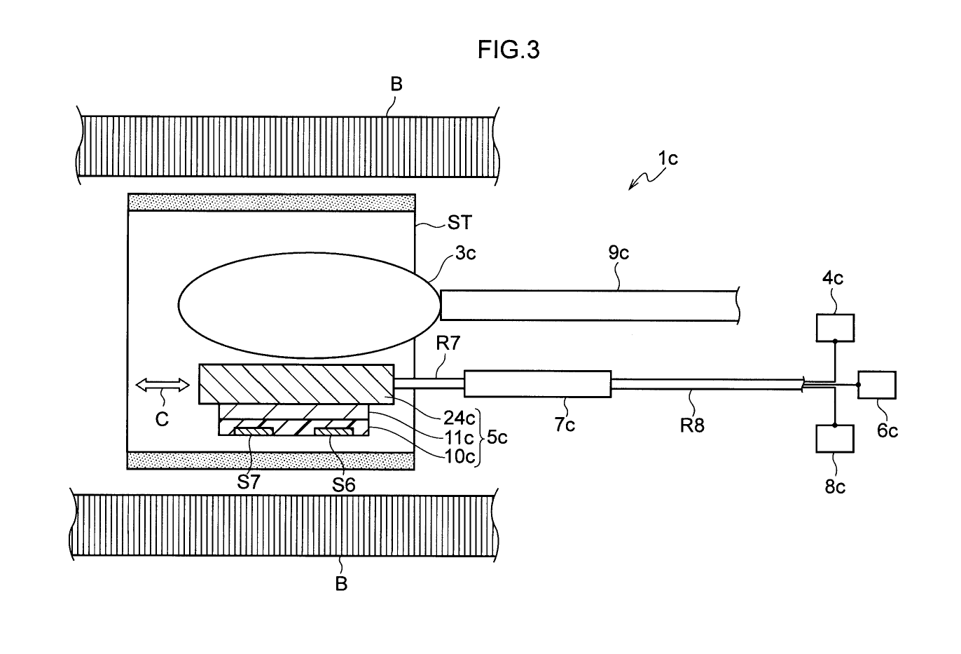

In a case where the medical instrument of the present invention is a stent, the stent can be bonded to the inner wall of a blood vessel using the later-described device 1c shown in FIG. 3.

As shown in FIG. 1, the clamping part 2a includes the members 21a and 22a, and clamps the adherends T1 and T2 between the members 21a and 22a. The shape, the size and the like of the members 21a and 22a, and the shape, the size and the like of a surface of the member 22a that contacts the adherend, are not particularly restricted, as long as the adherends T1 and T2 can be clamped between the members 21a and 22a. The members 21a and 22a have the shape of, for example, a plate, a clip or forceps. The surface of the member 22a that contacts the adherend has, for example, a planar, curved, serrated or pinholder form. The material of the member 22a is not particularly restricted as long as it does not adhere to the adherends T1 an T2, and examples of the material include stainless steel, polyester, cellophane, TEFLON (registered trademark), dry collagen, polyvinyl chloride, polyethylene, polypropylene, silk and composite materials thereof.

The heat-generating unit 5a has the resin member 10a and the vibration part 11a which imparts vibration to the resin member 10a. The type of the resin member 10a is not particularly restricted, but is preferably at least one selected from the group consisting of polytetrafluoroethylene, polyvinylidene fluoride, polyvinyl fluoride, perfluoroalkoxy fluorine resin, tetrafluoroethylene/hexafluoropropylene copolymer, ethylene/tetrafluoroethylene copolymer and ethylene/chlorotrifluoroethylene copolymer. The resin member 10a may also contain therein a glass cloth, nylon threads or the like.

In the heat-generating unit 5a, the surface of the resin member 10a that contact the adherend is, for example, planar, concave, convex or undulated. Further, the resin member 10a has a thickness of preferably 1 to 50 mm, more preferably 2 to 10 mm, in a direction perpendicular to the surface that contacts the adherend. The resin member 10a may be formed of a core, being made of an inorganic material such as ceramic, glass or glass ceramic or an organic material such as carbon fiber, polyether ether ketone resin (PEEK), polyamide or polyimide, and a resin layer of polytetrafluoroethylene or the like being formed on the core. In a case where the resin member 10a includes a core, the thickness of the resin member 10a refers to the total thickness of the core and a resin layer.

The vibration part 11a is not particularly restricted as long as it is a vibration source capable of applying vibration to the resin member 10a, and as the vibration part 11a, an electric motor, ultrasonic motor, piezoelectric element, small speaker or the like can be employed. Although the frequency of the vibration applied to the vibration member 10a depends on the type of the resin constituting the resin member 10a, it is preferably 1 to 100 kHz, more preferably 12 k to 50 kHz. In the present embodiment, the vibration part 11a imparts vibration to the resin member 10a in a direction perpendicular to the plane at which the vibration part 11a contacts the resin member 10a.

As shown in FIG. 1, the member 22a is attached to an arm AR1 via a rod R1 in such a manner that the member 22a can pivot about a shaft member G1. As shown in FIG. 1, the arm AR1 is provided with the pressing part 3a for allowing the member 22a to pivot. The pressing part 3a has an electric motor, an ultrasonic motor, a piezoelectric element or the like as a power source for allowing the member 22a to pivot, and presses the member 22a in a direction toward the member 21a by allowing the member 22a to pivot. Alternatively, the member 22a may be pressed in a direction toward the member 21a by connecting an end of a wire to the member 22a and externally pulling the other end of the wire.

As shown in FIG. 1, on the surface of the member 22a that contacts the adherend, a sensor S1 which detects the clamping force exerted by the clamping part 2a (that is, the pressure applied to the adherends T1 and T2 clamped by the clamping part 2a) is provided. The sensor S1 and the pressing part 3a are electrically connected to the clamping force control unit 4a which, based on the pressure and the like detected by the sensor S1, controls the pressure applied by the pressing part 3a in such a manner that the clamping force exerted by the clamping part 2a (that is, the pressure applied to the adherends T1 and T2 clamped by the clamping part 2a) is 9×10² to 1×10⁵ N/m² (preferably 1×10⁴ to 5×10⁴ N/m²).

In the heat-generating unit 5a, a sensor S2 which detects the temperatures of the adherends T1 and T2 is provided on the adherend-contacting surface of the resin member 10a. The sensor S2 and the heat-generating unit 5a are electrically connected to the heat generation control unit 6a which controls, based on the temperatures and the like detected by the sensor S2, heat generation of the heat-generating unit 5a in such a manner that the adherends T1 and T2 clamped by the clamping part 2a have a temperature of 60 to 140° C. (preferably 80 to 110° C.). Although the sensor S2 directly detects the temperature of the adherend T1, since the heat applied to the adherend T1 is transmitted to the adherend T2 and the temperature of the adherend T1 is affected by the temperature of the adherend T2, the sensor S2 can also detect the temperature of the adherend T2 based on the changes in the temperature and the like of the adherend T1.

As shown in FIG. 1, the member 21a is attached to the vibration unit 7a via a rod R2, and the vibration unit 7a is attached to an arm AR2. As a source for generating microvibration, the vibration unit 7a has a vibration element such as an ultrasonic oscillator, a micromotor or a magnetic body (in a case where a magnetic body is used, a variable magnetic field is externally applied). The microvibration generated by the vibration unit 7a is transmitted to the member 21a via the rod R2 which is a vibration transmitting member. The microvibration transmitted to the member 21a is then transmitted to the adherends T1 and T2 via the heat-generating unit 5a, which is configured integrally with the member 21a. The direction of the vibration applied to the member 21a is not particularly restricted; however, in the present embodiment, it is substantially parallel to the contact surface of the adherends T1 and T2 (the direction indicated by an arrow A in FIG. 1). To the vibration unit 7a, the vibration control unit 8a, which controls microvibration generated by the vibration unit 7a, is electrically connected. The vibration control unit 8a controls microvibration generated by the vibration unit 7a in such a manner that the frequency of the microvibration of the adherends T1 and T2 clamped by the clamping part 2a is 1 to 100 kHz (preferably 10 to 60 kHz). Further, the vibration control unit 8a also controls microvibration generated by the vibration unit 7a in such a manner that the amplitude of the vibration of the adherends T1 and T2 clamped by the clamping part 2a is less than 100 μm, preferably less than 20 μm. Here, the lower limit of the amplitude of the microvibration is usually 0.1 μm, preferably 0.2 μm. In a case where the adherends T1 and T2 clamped by the clamping part 2a are vibrated at an amplitude of less than 100 μm, the size of the device 1a can be reduced since a compact vibration element can be used and there is no need to provide a horn.

As shown in FIG. 1, the arm AR1 is fixed to the arm AR2, which is connected to a grip (not shown), a catheter (not shown), a guide wire (not shown) or the like.

The device 1a bonds the adherends T1 and T2 in the following manner.

The clamping part 2a clamps the adherends T1 and T2 being in contact with each other. During clamping, the clamping force exerted by the clamping part 2a is controlled by the clamping force control unit 4a, and a pressure of 9×10² to 1×10⁵ N/m² (preferably 1×10⁴ to 5×10⁴ N/m²) is applied to the adherends T1 and T2 clamped by the clamping part 2a.

Further, heat generated by the heating element 5a is transmitted to the adherends T1 and T2 via the surface of the resin member 10a that contacts the adherend, and the adherends T1 and T2 are heated. During the process, heat generation of the heating element 5a is controlled by the heat generation control unit 6a, so that the adherends T1 and T2 clamped by the clamping part 2a are heated to a temperature of 60 to 140° C. (preferably 80 to 110° C.). The heat generated by the heating element 5a is initially applied to the adherend T1; however, since the adherends T1 and T2 are in contact with each other, the heat applied to the adherend T1 is transmitted to the adherend T2, and the adherend T2 is also heated.

Further, microvibration generated by the vibration unit 7a is transmitted to the heat-generating unit 5a, which is configured integrally with the member 21a, via the rod R2 which is a vibration transmitting member. The vibration generated by the vibration unit 7a is controlled by the vibration control unit 8a, and the adherends T1 and T2 clamped by the clamping part 2a vibrate at a frequency of 1 to 100 kHz (preferably 10 to 60 kHz). The microvibration generated by the vibration unit 7a is initially applied to the adherend T1; however, since the adherends T1 and T2 are in contact with each other, the vibration applied to the adherend T1 is transmitted to the adherend T2, and the adherend T2 is also vibrated. The direction of the vibration applied to the adherends T1 and T2 is not particularly restricted; however, in the present embodiment, it is substantially parallel to the contact surface of the adherends T1 and T2 (the direction indicated by the arrow A in FIG. 1).

Accordingly, the adherends T1 and T2 clamped by the clamping part 2a are in contact with each other and subjected to a pressure of 9×10² to 1×10⁵ N/m² (preferably 10⁴ to 5×10⁴ N/m²), a temperature of 60 to 140° C. (preferably 80 to 110° C.) and a vibration having a frequency of 1 to 100 kHz (preferably 10 to 60 kHz). The time for application of the pressure, the temperature and the vibration as mentioned above to the adherends T1 and T2 is usually 2 to 240 seconds, preferably 10 to 120 seconds. In that case, the adherends T1 and T2 are bonded quickly and strongly. Furthermore, when the adherends T1 and T2 are subjected to the pressure, temperature and vibration as mentioned above, the damage to the adherends T1 and T2 is suppressed.

In the first embodiment, the vibration unit 7a is configured to vibrate the member 21a; however, the vibration unit 7a may also be provided between the pressing part 3a and the member 22a, and configured to vibrate the member 22a.

Further, in the first embodiment, the device may have a constitution in which the heat-generating unit 5a and the heat generation control unit 6a serve as the vibration unit 7a and the vibration control unit 8a, respectively, and transmit the vibration which has been applied to the resin member 10a by the vibration part 11a to the adherends T1 and T2.

In the first embodiment, a heat-generating device is formed from the heat-generating unit 5a and the heat generation control unit 6a. The heat-generating device according to the first embodiment is different from conventional heat-generating devices, such as electric heaters, in that it is not necessary to supply an electric current to the resin member 10a contacting the adherend T1 in order to allow the heat-generating unit 5a to generate heat. Therefore, the biological tissue-bonding device according to the first embodiment is effective for adherends that are susceptible to an electric field (for example, brain tissues such as cranial nerve).

### Second Embodiment

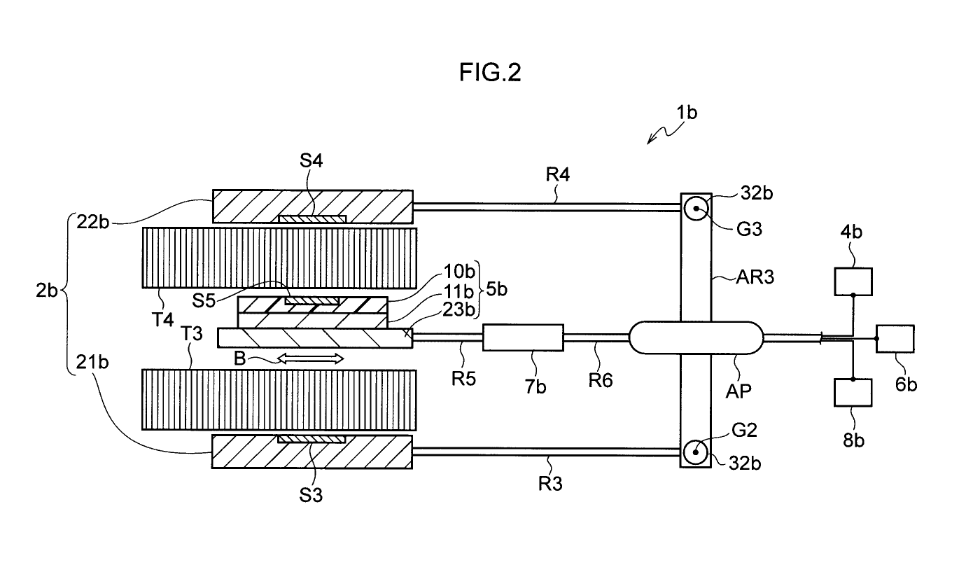

The biological tissue-bonding device 1b according to the second embodiment is a device for bonding adherends T3 and T4. As shown in FIG. 2, the biological tissue-bonding device 1b includes a clamping part 2b which clamps the adherends T3 and T4 between members 21b and 22b; a heat-generating unit 5b being interposed between the adherends T3 and T4, and having, on one side of a member 23b, a vibration part 11b and a resin member 10b in this order from the member 23b side; a pressing part 31b which presses the member 21b in a direction toward the member 22b; a pressing part 32b which presses the member 22b in a direction toward the member 21b; a clamping force control unit 4b which controls pressure (clamping force) exerted by the pressing parts 31b and 32b; a heat generation control unit 6b which controls, by controlling the vibration generated by the vibration part 11b, heat generation of the heat-generating unit 5b in such a manner the heat-generating unit 5b has a prescribed temperature; a vibration unit 7b which generates microvibration; and a vibration control unit 8b which controls the microvibration generated by the vibration unit 7b.

The type of the adherends T3 and T4 is not particularly restricted. It is possible that both of the adherends T3 and T4 are a biological tissue, or that either one of them is a biological tissue while the other is a material capable of being bonded to a biological tissue. Specific examples of the biological tissue and the biological tissue-bonding material are the same as those described above. The thicknesses of the adherends T3 and T4 (the thicknesses in a direction perpendicular to the contact surface of the adherends T3 and T4) are not particularly restricted, but are usually 0.01 to 10 mm, preferably 0.1 to 5 mm. The device 1b according to the second embodiment is suitable for bonding adherends having a relatively large thickness.

As shown in FIG. 2, the clamping part 2b has the members 21b and 22b, and clamps the adherends T3 and T4 between the members 21b and 22b. The shape, the size and the like of the members 21b and 22b, as well as the shape, the size and the like of the surface of the members 21b and 22b that contacts the adherend are not particularly restricted, as long as the adherends T3 and T4 can be clamped between the members 21b and 22b. The members 21b and 22b have the shape of, for example, a plate, a clip or forceps. The surface of the members 21b and 22b that contacts the adherend is, for example, planar, curved, serrated or in a pinholder form. The material of the members 21b and 22b is not particularly restricted as long as it does not adhere to the adherends T3 an T4, and examples of the material include stainless steel, polyester, cellophane, TEFLON (registered trademark), dry collagen, polyvinyl chloride, polyethylene, polypropylene, silk and composite materials thereof.

As shown in FIG. 2, the member 21b is attached to an arm AR3 via a rod R3 in such a manner that the member 21b can pivot about a shaft member G2, and the member 22b is attached to the arm AR3 via a rod R4 in such a manner that the member 22b can pivot about a shaft member G3. As shown in FIG. 2, the arm AR3 is provided with the pressing part 31b for pivoting the member 21b and the pressing part 32b for pivoting the member 22b. Each of the pressing parts 31b and 32b has an electric motor, an ultrasonic motor, a piezoelectric element or the like as a power source for pivoting the members 21b and 22b, respectively. The pressing part 31b presses the member 21b in a direction toward the member 22b by allowing the member 21b to pivot, and the pressing part 32b presses the member 22b in a direction toward the member 21b by allowing the member 22b to pivot. Alternatively, the member 21b may be pressed in a direction toward the member 22b by connecting an end of a wire to the member 21b and externally pulling the other end of the wire, or the member 22b may be pressed in a direction toward the member 21b by connecting an end of a wire to the member 22b and externally pulling the other end of the wire.

As shown in FIG. 2, on the surface of the members 21b and 22b that contacts the adherend, sensors S3 and S4, which detect a clamping force exerted by the clamping part 2b (that is, a pressure applied to the adherends T3 and T4 clamped by the clamping part 2b, respectively), are provided, respectively. The sensors S3 and S4 and the pressing parts 31b and 32b are electrically connected to the clamping force control unit 4b which controls, based on the pressures and the like detected by the sensors S3 and S4, the pressure exerted by the pressing parts 31b and 32b in such a manner that the clamping force exerted by the clamping part 2b (that is, the pressure applied to the adherends T3 and T4 clamped by the clamping part 2b) is 9×10² to 1×10⁵ N/m² (preferably 1×10⁴ to 5×10⁴ N/m²). Typically, the clamping force control unit 4b controls the pressure exerted by the pressing parts 31b and 32b in such a manner that the pressure exerted by the pressing part 31b and applied to the member 21b in a direction toward the member 22b and the pressure exerted by the pressing part 32b and applied to the member 22b in a direction toward the member 21b are equal to each other.

The heat-generating unit 5b has a configuration in which, on one side of the member 23b, the vibration part 11b and the resin member 10b are provided in this order from the member 23b side. The shape, the size and the like of the member 23b, and the shape, the size and the like of a surface of the member 23b that contacts the adherend T3 are not particularly restricted, as long as the member 23b can be interposed between the adherends T3 and T4 being in contact with each other, and the vibration part 11b and the resin member 10b can be provided on one side of the member 23b. The member 23b has the shape of, for example, a plate or a rod. The surface of the member 23b that contacts the adherend T3 has, for example, a planar, curved, serrated or pinholder form.

The type of the resin member 10b is not particularly restricted, but is preferably at least one selected from the group consisting of polytetrafluoroethylene, polyvinylidene fluoride, polyvinyl fluoride, perfluoroalkoxy fluorine resin, tetrafluoroethylene/hexafluoropropylene copolymer, ethylene/tetrafluoroethylene copolymer and ethylene/chlorotrifluoroethylene copolymer. The resin member 10b may also contain therein a glass cloth, nylon threads or the like. The surface of the member 10b that contacts the adherend is, for example, planar, concave, convex or undulated. Further, the resin member 10b has a thickness of preferably 1 to 50 mm, more preferably 2 to 10 mm, in a direction perpendicular to the surface that contacts the adherend T4. The resin member 10b may be formed of a core, being made of an inorganic material such as ceramic, glass or glass ceramic or an organic material such as carbon fiber, polyether ether ketone resin (PEEK), polyamide or polyimide, and a resin layer of polytetrafluoroethylene or the like being formed on the core. In a case where the resin member 10b includes a core, the thickness of the resin member 10b refers to the total thickness of the core and a resin layer.

The vibration part 11b is not particularly restricted as long as it is a vibration source capable of applying vibration to the resin member 10b, and a piezoelectric element, a small speaker or the like can be used as the vibration part 11b. Although the frequency of the vibration to be applied to the resin member 10b depends on the type of the resin that constitutes the resin member 10b, it is preferably 1 to 100 kHz, more preferably 12 k to 50 kHz. In the present embodiment, the vibration part 11b imparts vibration to the resin member 10b in a direction perpendicular to a plane at which the vibration part 11b contacts the resin member 10b.

On the surface of the resin member 10b that contacts the adherend, a sensor S5 which detects the temperature of the adherend T4 is provided. The sensor S5 and the heat-generating unit 5b are electrically connected to the heat generation control unit 6b which controls, based on the temperature and the like detected by the sensor S5, heat generation of the heat-generating unit 5b in such a manner that the adherend T4 clamped by the clamping part 2b has a temperature of 60 to 140° C. (preferably 80 to 110° C.).

As shown in FIG. 2, the member 23b is attached to the vibration unit 7b via a rod R5, the vibration unit 7b is attached to an adaptor AP via a rod R6, and the adaptor AP is attached to the arm AR3. As a source for generating microvibration, the vibration unit 7b has a vibration element such as an ultrasonic oscillator, a micromotor or a magnetic body (in a case where a magnetic body is used, a variable magnetic field is externally applied). The microvibration generated by the vibration unit 7b is transmitted to the member 23b via the rod R5, which is a vibration transmitting member, and vibrates the heat-generating unit 5b. The direction of the vibration applied to the heat-generating unit 5b is not particularly restricted; however, in the present embodiment, it is substantially parallel to the contact surface of the adherends T3 and T4 (the direction indicated by an arrow B in FIG. 2). To the vibration unit 7b, the vibration control unit 8b, which controls the microvibration generated by the vibration unit 7b, is electrically connected. The vibration control unit 8b controls the vibration generated by the vibration unit 7b in such a manner that the frequency of the microvibration of the adherends T3 and T4, being clamped by the clamping part 2b, is 1 to 100 kHz (preferably 10 to 60 kHz). Further, the vibration control unit 8b also controls the microvibration generated by the vibration unit 7b in such a manner that the amplitude of the vibration of the adherends T3 and T4, being clamped by the clamping part 2b, is less than 100 μm, preferably less than 20 μm. The lower limit of the amplitude of the microvibration is usually 0.1 μm, preferably 0.2 μm. In a case where the adherends T3 and T4 clamped by the clamping part 2b are vibrated at an amplitude of less than 100 μm, the size of the biological tissue-bonding device 1b can be reduced since a compact vibration element can be used and there is no need to provide a horn. It is noted that although the microvibration generated by the vibration unit 7b is transmitted to the adaptor AP via the rod R6, since the adaptor AP has a mechanism capable of absorbing microvibration (for example, a microvibration-absorbing mechanism utilizing an elastic member), the microvibration is not transmitted to the arm AR3. The adaptor AP is connected to a grip (not shown), a catheter (not shown), a guide wire (not shown) or the like.

The biological tissue-bonding device 1b bonds the adherends T3 and T4 in the following manner.

The clamping part 2b clamps the adherends T3 and T4 being in contact with each other, and the heat-generating unit 5b is interposed therebetween. The clamping force exerted by the clamping part 2b is controlled by the clamping force control unit 4b, and a pressure of 9×10² to 1×10⁵ N/m² (preferably 1×10⁴ to 5×10⁴ N/m²) is applied to the adherends T3 and T4 clamped by the clamping part 2b.

Further, the heat generated by the heat-generating unit 5b is transmitted to the adherends T4 and T3 via the surface of the resin member 10b that contacts the adherend, and the adherends T3 and T4 are heated. By controlling the heat generated by the heating element 5b by the heat generation control unit 6b, the adherends T3 and T4 clamped by the clamping part 2b are heated to a temperature of 60 to 140° C. (preferably 80 to 110° C.).

Further, the microvibration generated by the vibration unit 7b is transmitted to the heat-generating unit 5b via the rod R5, which is a vibration transmitting member. Since the heat-generating unit 5b is interposed between the adherends T3 and T4, the vibration of the heat-generating unit 5b is transmitted to the adherends T3 and T4. By controlling the vibration generated by the vibration unit 7b by the vibration control unit 8b, the adherends T3 and T4 clamped by the clamping part 2b are vibrated at a frequency of 1 to 100 kHz (preferably 10 to 60 kHz). The direction of the vibration to be applied to the adherends T3 and T4 is not particularly restricted; however, in the present embodiment, it is substantially parallel to the contact surface of the adherends T3 and T4 (the direction indicated by the arrow B in FIG. 1).

Accordingly, the adherends T3 and T4, being clamped by the clamping part 2b, are in contact with each other and subjected to a pressure of 9×10² to 1×10⁵ N/m² (preferably 1×10⁴ to 5×10⁴ N/m²), a temperature of 60 to 140° C. (preferably 80 to 110° C.) and a vibration having a frequency of 1 to 100 kHz (preferably 10 to 60 kHz). The time for application of the pressure, the temperature and the vibration as mentioned above to the adherends T3 and T4 is usually 2 to 240 seconds, preferably 10 to 120 seconds. In that case, the adherends T3 and T4 are bonded quickly and strongly. Furthermore, when the pressure, the temperature and the vibration as mentioned above are applied to the adherends T3 and T4, the damage to the adherends is suppressed. It should be noted that the portion of the adherends T3 and T4 not being in contact with each other, due to the presence of the heat-generating unit 5b interposed therebetween, is not bonded.

In the second embodiment, the vibration unit 7b is configured to vibrate the heat-generating unit 5b; however, the vibration unit 7b may also be configured to vibrate at least one of the members 21b and 22b.

Further, in the second embodiment, the device may have a constitution in which the heat-generating unit 5b and the heat generation control unit 6b also serve as the vibration unit 7b and the vibration control unit 8b, and transmit the vibration applied to the resin member 10b by the vibration part 11b to the adherends T3 and T4.

In the second embodiment, a heat-generating device includes the heat-generating unit 5b and the heat generation control unit 6b. The heat-generating device according to the second embodiment is different from conventional heat-generating devices, such as electric heaters, in that it is not necessary to supply an electric current to the resin member 10b that contacts the adherend T4 in order to allow the heat-generating unit 5b to generate heat. Therefore, the biological tissue-bonding device according to the second embodiment is effective for adherends that are susceptible to an electric field (for example, brain tissues such as cranial nerve).

### Third Embodiment

The biological tissue-bonding device 1c according to the third embodiment is a biological tissue-bonding device for bonding a stent ST, being inserted into a blood vessel B, to an inner wall of the blood vessel B. As shown in FIG. 3, the biological tissue-bonding device 1c includes a heat-generating unit 5c that has, on one side of a member 24c, a vibration part 11c and a resin member 10c being provided in this order from the member 24c side; a balloon 3c which presses the heat-generating unit 5c in a direction toward the inter wall of the blood vessel B; a pressure control unit 4c which controls a pressure exerted by the balloon 3c; a heat generation control unit 6c which controls heat generation of the heat-generating unit 5c; a vibration unit 7c which generates microvibration; and a vibration control unit 8c which controls the microvibration generated by the vibration unit 7c.

The surface of the stent ST is coated with a material capable of being bonded to a biological tissue, such as wet collagen, polyurethane, vinylon, gelatin or a composite material thereof.

As shown in FIG. 3, the balloon 3c is in communication with a balloon catheter 9c, and by injecting a fluid into the balloon 3c, the balloon 3c is inflated to expand a stenotic part of the blood vessel B and press the heat-generating unit 5c in a direction toward the inter wall of the blood vessel B. In the heat-generating unit 5c, a sensor S6, which detects the pressure applied to the stent ST and the blood vessel B, is provided on the surface of the resin member 10c that contacts the stent. The sensor S6 and a device for injecting a fluid into the balloon 3c (not shown) are electrically connected to the pressure control unit 4c which controls, based on the pressure and the like detected by the sensor S6, the pressure exerted by the balloon 3c in such a manner that the pressure applied to the stent ST and the blood vessel B is 9×10² to 1×10⁵ N/m² (preferably 1×10⁴ to 5×10⁴ N/m²).

The heat-generating unit 5c has a configuration in which, on one side of the member 24c, the vibration part 11c and the resin member 10c are provided in this order from the member 24c side. The shape, the size and the like of the member 24c are not particularly restricted, as long as the member 24c can be inserted into the stent ST and the vibration part 11c and the resin member 10c can be provided on one side of the member 24c. The member 24c has a shape of, for example, a plate or a rod. The material of the member 24c is not particularly restricted as long as it does not adhere to the stent ST, and examples of the material include stainless steel, polyester, cellophane, TEFLON (registered trademark), polyvinyl chloride, polyethylene, polypropylene, silk, aramid resin, polyether ether ketone resin, silicone resin, polycarbonate resin and composite materials thereof.

The type of the resin member 10c is not particularly restricted, but is preferably at least one selected from the group consisting of polytetrafluoroethylene, polyvinylidene fluoride, polyvinyl fluoride, perfluoroalkoxy fluorine resin, tetrafluoroethylene/hexafluoropropylene copolymer, ethylene/tetrafluoroethylene copolymer and ethylene/chlorotrifluoroethylene copolymer. The resin member 10c may also contain therein a glass cloth, nylon threads or the like. The surface of the member 10c that contacts the stent is, for example, planar, concave, convex or undulated. The resin member 10c has a thickness of preferably 1 to 50 mm, more preferably 2 to 10 mm, in a direction perpendicular to the surface that contacts the stent. The resin member 10c may be formed of a core, being made of an inorganic material such as ceramic, glass or glass ceramic or an organic material such as carbon fiber, PEEK, polyamide or polyimide, and a resin layer of polytetrafluoroethylene or the like being formed on the core. In a case where the resin member 10c includes a core, the thickness of the resin member 10c refers to the total thickness of the core and a resin layer.

The vibration part 11c is not particularly restricted as long as it is a vibration source capable of applying vibration to the resin member 10c, and a piezoelectric element, a small speaker or the like can be used as the vibration part 11c. Although the frequency of the vibration applied to the resin member 10c depends on the type of the resin that constitutes the resin member 10c, it is preferably 1 to 100 kHz, more preferably 12 k to 50 kHz. In the present embodiment, the vibration part 11c imparts vibration to the resin member 10c in a direction perpendicular to the plane at which the vibration part 11c contacts the resin member 10c.

As shown in FIG. 3, on the surface of the resin member 10c that contacts the stent, a sensor S7, which detects the temperatures of the stent ST and the inner wall of the blood vessel B, is provided. The sensor S7 and the heat-generating unit 5c are electrically connected to the heat generation control unit 6c which controls, based on the temperature and the like detected by the sensor S7, heat generation of the heat-generating unit 5c in such a manner that the stent ST and the inner wall of the blood vessel B have a temperature of 60 to 140° C. (preferably 80 to 110° C.). Although the sensor S7 directly detects the temperature of the stent ST, since the heat applied to the stent ST is transmitted to the inner wall of the blood vessel B and the temperature of the stent ST is affected by the temperature of the inner wall of the blood vessel B, the sensor S7 can also detect the temperature of the inner wall of the blood vessel B, based on the changes in the temperature and the like of the stent ST.

As shown in FIG. 3, the member 24c is attached to the vibration unit 7c via a rod R7, and the vibration unit 7c is attached to a rod R8. As a source for generating microvibration, the vibration unit 7c has a vibration element such as an ultrasonic oscillator, a micromotor or a magnetic body (in a case where a magnetic body is used, a variable magnetic field is externally applied). The microvibration generated by the vibration unit 7c is transmitted to the member 24c via the rod R7, which is a vibration transmitting member, and vibrates the heat-generating unit 5c. The direction of the vibration to be applied to the heat-generating unit 5c is not particularly restricted; however, in the present embodiment, it is substantially parallel to the contact surface of the stent ST and the inner wall of the blood vessel B (the direction indicated by an arrow C in FIG. 3). To the vibration unit 7c, the vibration control unit 8c, which controls the microvibration generated by the vibration unit 7c, is electrically connected. The vibration control unit 8c controls the vibration generated by the vibration unit 7c in such a manner that the frequency of the microvibration of the stent ST and the inner wall of the blood vessel B is 1 to 100 kHz (preferably 10 to 60 kHz). The vibration control unit 8c also controls the microvibration generated by the vibration unit 7c in such a manner that the amplitude of the vibration of the stent ST and the inner wall of the blood vessel B is less than 100 μm, preferably less than 20 μm. The lower limit of the amplitude of the microvibration is usually 0.1 μm, preferably 0.2 μm. In a case where the stent ST and the inner wall of the blood vessel B are vibrated at an amplitude of less than 100 μm, the size of the device 1c can be reduced since a compact vibration element can be used and there is no need to provide a horn. The rod R8 is connected to a grip (not shown), a catheter (not shown), a guide wire (not shown) or the like.

The device 1c bonds the stent ST with the inner wall of the blood vessel B in a manner as described below.

When a fluid is injected into the balloon 3c via the balloon catheter 9c, the balloon 3c is inflated to expand a stenotic part of the blood vessel B and press the heat-generating unit 5c in a direction toward the inner wall of the blood vessel B, thereby pressing the stent ST against the inner wall of the blood vessel B. In this way, the stent ST is brought into contact with the inter wall of the blood vessel B. The pressure exerted by the balloon 3c is controlled by the pressure control unit 4c, and a pressure of 9×10² to 1×10⁵ N/m² (preferably 1×10⁴ to 5×10⁴ N/m²) is applied to the stent ST and the inner wall of the blood vessel B.

The heat generated by the heat-generating unit 5c is transmitted to the stent ST and the inner wall of the blood vessel B via the surface of the resin member 10c that contacts the stent, and the stent ST and the inner wall of the blood vessel B are heated. During heating, the heat generation of the heating element 5c is controlled by the heat generation control unit 6c, and the stent ST and the inner wall of the blood vessel B are heated to a temperature of 60 to 140° C. (preferably 80 to 110° C.). The heat generated by the heating element 5c is initially applied to the stent ST, but since the stent ST is in contact with the inner wall of the blood vessel B, the heat applied to the stent ST is transmitted to the inner wall of the blood vessel B and heats the inner wall of the blood vessel B as well.

Further, the microvibration generated by the vibration unit 7c is transmitted to the heat-generating unit 5c via the rod R7, which is a vibration transmitting member. Since the heat-generating unit 5c contacts the stent ST, vibration of the heat-generating unit 5c is transmitted to the stent ST and the inner wall of the blood vessel B. The vibration generated by the vibration unit 7c is controlled by the vibration control unit 8c, and vibrates the stent ST and the inner wall of the blood vessel B at a frequency of 1 to 100 kHz (preferably 10 to 60 kHz). The microvibration generated by the vibration unit 7c is initially applied to the stent ST, but since the stent ST is in contact with the inner wall of the blood vessel B, the vibration applied to the stent ST is transmitted to the inner wall of the blood vessel B and vibrates the inner wall of the blood vessel B as well. The direction of the vibration applied to the stent ST and the inner wall of the blood vessel B is not particularly restricted; however, in the present embodiment, it is substantially parallel to the contact surface of the stent ST and the inner wall of the blood vessel B (the direction indicated by the arrow C in FIG. 3).

Accordingly, the stent ST and the inner wall of the blood vessel B are in contact with each other, and are subjected to a pressure of 9×10² to 1×10⁵ N/m² (preferably 1×10⁴ to 5×10⁴ N/m²), a temperature of 60 to 140° C. (preferably 80 to 110° C.) and a vibration having a frequency of 1 to 100 kHz (preferably 10 to 60 kHz). The time for application of the pressure, the temperature and the vibration to the stent ST and the inner wall of the blood vessel B are usually 2 to 240 seconds, preferably 10 to 120 seconds. In this way, the stent ST is bonded with the inner wall of the blood vessel B quickly and strongly. Furthermore, when the stent ST and the inner wall of the blood vessel B are subjected to the pressure, the temperature and the vibration as mentioned above, the damage to the stent ST and the inner wall of the blood vessel B is suppressed.

In the third embodiment, the device may have a constitution in which the heat-generating unit 5c and the heat generation control unit 6c also serve as the vibration unit 7c and the vibration control unit 8c, and transmit the vibration applied to the resin member 10c by the vibration part 11c to the stent ST and the blood vessel B.

In the third embodiment, a heat-generating device includes the heat-generating unit 5c and the heat generation control unit 6c. The heat-generating device according to the third embodiment is different from conventional heat-generating devices, such as electric heaters, in that it is not necessary to supply an electric current to the resin member 10c that contacts the stent ST in order to allow the heat-generating unit 5c to generate heat. Therefore, the biological tissue-bonding device according to the third embodiment is effective for adherends that are susceptible to an electric field (for example, brain tissues such as cranial nerve).

### EXAMPLES

### Test Example 1

The present test example was carried out in order to verify the state of a resin member being heated upon application of vibration thereto.

On a 5 mm×5 mm ceramic plate having a thickness of 1.75 mm (trade name: MICRO CERAMIC HEATER, manufactured by Sakaguchi E.H Voc Corp.), which was used as a core, a PTFE (polytetrafluoroethylene) fluoroglass adhesive tape (trade name: CHUKOH FLO AGF-110, manufactured by Chukoh Chemical Industries, Ltd.) was wound three times to form a resin layer of 0.4 mm in thickness on both sides of the ceramic plate, thereby obtaining sample 1. As a control, a ceramic plate without a PTFE fluoroglass adhesive tape was used as sample 2.

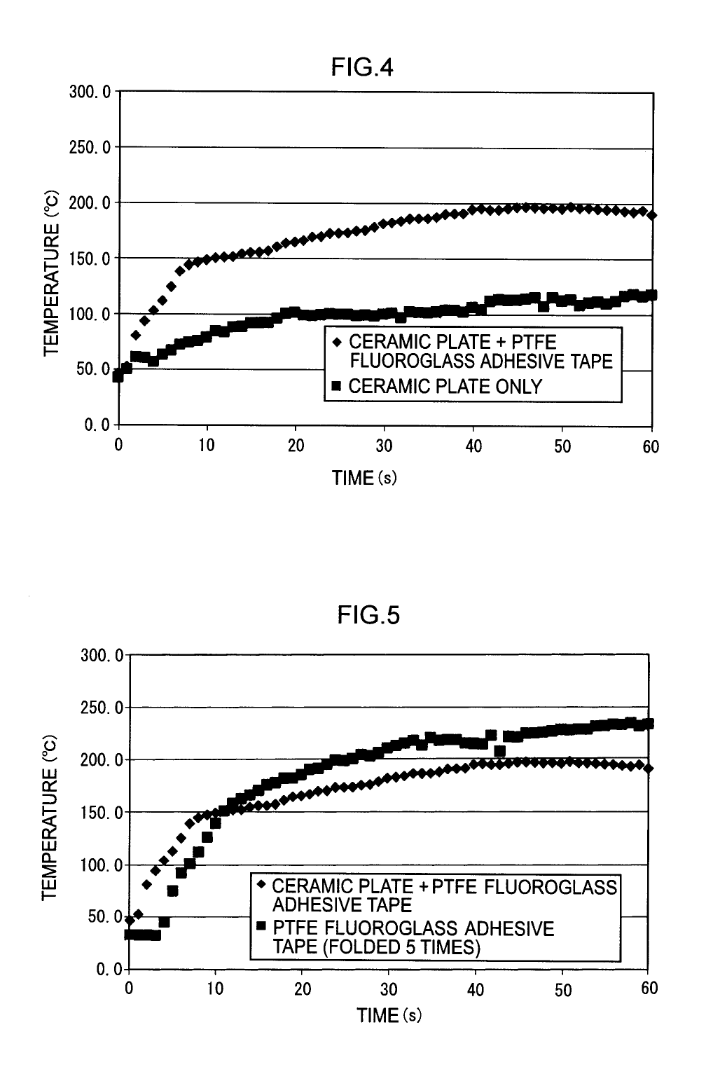

The sample 1 was placed between a stainless steel plate member (7 mm in lateral width, 5 mm in longitudinal width, 3 mm in thickness) and a vibration-generating apparatus (trade name: NANO VIBRATOR, manufactured by Miwatec Co., Ltd., the portion which contacts the sample to apply vibration had a size of 5 mm×5 mm) and clamped at a pressure of 0.4 N/mm². A vibration (longitudinal vibration) having a vibration width of 5 μm and a frequency of 12 kHz was applied by the vibration-generating apparatus to the resin layer in a thickness direction of the ceramic plate. Changes in the temperature at the portion of the sample 1 to which the vibration was applied were measured by thermography (trade name: THERMOTRACER, manufactured by NEC Corporation). FIG. 4 shows the temperature change with respect to the time for applying vibration. Further, vibration having the same vibration width and the same frequency was applied to the sample 2, and the temperature change thereof was measured in the same manner. The measurement results are shown in FIG. 4. As shown in FIG. 4, although an increase in the temperature of the sample 2 was observed as the time for applying vibration lapsed, it was less than an increase in the temperature of the sample 1.

A PTFE fluoroglass adhesive tape was folded five times without a core, thereby obtaining sample 3 of 1.3 mm in thickness formed of ten layers of the PTFE fluoroglass adhesive tape. The sample 3 was evaluated in the same manner as the sample 1. The results are shown in FIG. 5 together with the results of the sample 1. The sample 3, being formed only of the PTFE fluoroglass adhesive tape, also generated heat in a similar manner to the sample 1 including a ceramic plate as a core. In addition, from the observation by thermography, it was revealed that heat was generated inside the sample 3, rather than at a region at which the sample 3 was in contact with a portion at which the vibration-generating apparatus contacted to apply vibration to the sample 3. This fact suggests that the heat generation by application of vibration to the resin member is caused by a mechanism different from friction.

In addition, samples 4 to 6 were prepared in the same manner as the sample 3, except that the number of layers of the PTEF fluoroglass adhesive tape was changed to 10 (thickness: 1.3 mm), 15 (thickness: 2 mm) and 20 (thickness: 2.8 mm), respectively, and the thus obtained samples 4 to 6 were evaluated in the same manner as the sample 1. The temperatures measured after 60 seconds of application of vibration of the samples 4 to 6 were 132° C., 117° C. and 90° C., respectively.

Sample 7 (thickness: 0.4 mm) and sample 8 (thickness: 0.4 mm) were prepared in the same manner as the sample 3, except that a PTFE adhesive tape (trade name: CHUKOH FLO ASF-110, manufactured by Chukoh Chemical Industries, Ltd.; folded three times) and TEFLON (registered trademark) seal tape (trade name: TEFLON (registered trademark) SEAL TAPE, manufactured by TGK; folded 10 times) were used in place of the PTFE fluoroglass adhesive tape, respectively. The thus obtained samples 7 and 8 were evaluated in the same manner as the sample 1. The temperatures of the samples 7 and 8 as measured 60 seconds after the application of vibration were both 210° C.

Samples 9 and 10 were prepared using a PTFE plate (trade name: PTFE SHEET, manufactured by Sanplatec Co., Ltd.) and a PFA (tetrafluoroethylene/perfluoroalkylvinylether copolymer) plate (trade name: PFA SHEET, manufactured by Nichias Corporation), both having a thickness of 2 to 3 mm, in place of the PTFE fluoroglass adhesive tape, respectively. The thus obtained samples 9 and 10 were evaluated in the same manner as the sample 1. The temperatures of the samples 9 and 10 as measured 60 seconds after the application of vibration were 150° C. and 160° C., respectively.

Samples 11 to 13 were prepared using a polyethylene terephthalate (PET) plate (manufactured by Sanplatec Co., Ltd.), a polymethyl methacrylate (PMMA) plate (manufactured by Sanplatec Co., Ltd.) and a polyvinyl chloride (PVC) plate (manufactured by Sanplatec Co., Ltd.), all having a thickness of 2 to 3 mm, in place of the PTFE fluoroglass adhesive tape, respectively. The thus obtained samples 11 to 13 were evaluated in the same manner as the sample 1. The temperature of the sample 11 as measured after reaching 40° C. in 5 seconds of application of vibration, and after 60 seconds of the application of vibration, was 120° C. The temperature of the sample 12 as measured after reaching 100° C. in 5 seconds of application of vibration, and after 60 seconds of application of vibration, was 145° C. The temperature of the sample 13 as measured after reaching 50° C. in 5 seconds of application of vibration, and after 60 seconds of application of vibration, was 150° C. If was confirmed that the samples 11 to 13, 60 seconds after the application of vibration, were melted and deformed by heat.

On a PTFE plate having a long side of 5 mm, a short side of 5 mm and a thickness of 2 mm (manufactured by Sanplatec Co., Ltd.), which was used as a core, a PTFE fluoroglass adhesive tape (trade name: CHUKOH FLO AGF-110, manufactured by Chukoh Chemical Industries, Ltd.) was wound three times to form a resin layer of 0.4 mm in thickness on both sides of the PTFE plate, thereby obtaining sample 14. The sample 14 was evaluated in the same manner as the sample 1. As a result, the temperature of the sample 14 60 seconds after the application of vibration was 260° C.

### Test Example 2

This test example is a bonding test of a biological tissue using the biological tissue-bonding device according to the present invention.

As a biological tissue material to be bonded, a porcine aorta was used. Adipose tissues were removed from the porcine aorta, and a portion having an average thickness of 1.0 to 1.5 mm was shaped into a size of 15×15 mm, thereby obtaining a tissue sample.

The bonding property of the biological tissue was examined with an ultrasonic scalpel (trade name: SONOPET, manufactured by Miwatec Co., Ltd.), and with the biological tissue-bonding device according to the first embodiment.

(Biological Tissue-Bonding Device)

Polytetrafluoroethylene (PTFE) was used as a resin member and a piezo drive was used as a vibration part. Vibration was applied to the resin member in a direction perpendicular to the surface of the vibration part that contacts the resin member. The vibration applied by the vibration part was set to have a frequency of 20 kHz and an amplitude of 5 μm. The temperature of the vascular tissue piece (adherend) at this time was 200° C. Further, the vibration applied by the vibration part was set to have a frequency of 20 kHz and an amplitude of 5 μm, the clamping force exerted by the clamping part was set to be 3.9×10⁴ N/m², and the time for press-bonding was set to be 30 seconds. Under these conditions, bonding of two vascular tissue pieces was attempted.

(Ultrasonic Scalpel)

Bonding of two vascular tissue pieces was attempted by applying vibration having a frequency of 55.5 kHz and an amplitude of 100 μm, at a temperature of 120° C. and a pressure of 3.9×10⁴ N/m², for a press-bonding time of 5 seconds.

As a result of the bonding experiment, it was possible to bond the biological tissues with the ultrasonic scalpel and the ultrasonic bonding apparatus.

However, while the ultrasonic scalpel was only able to bond thin aortae (having a thickness of approximately 0.5 mm), the ultrasonic bonding apparatus was able to bond relatively thick aortae (having a thickness of approximately 1.0 mm) as well.

### DESCRIPTION OF SYMBOLS

1a, 1b, 1c: biological tissue-bonding device
2a, 2b: clamping part
3a, 31b, 32b: pressing part
3c: pressing part (balloon)
4a, 4b: clamping force control unit
4c: pressure control unit
5a, 5b, 5c: heat-generating unit
6a, 6b, 6c: heat generation control unit
7a, 7b, 7c: vibration unit
8a, 8b, 8c: vibration control unit
10a, 10b, 10c: resin member
11a, 11b, 11c: vibration part
T1, T2, T3, T4: adherend (biological tissue or biological tissue-bonding material)
B: adherend (blood vessel)
ST: adherend (stent)

1. A biological tissue-bonding device for bonding a biological tissue which is a first adherend, and a biological tissue or a material capable of being bonded to a biological tissue which is a second adherend, the biological tissue-bonding device comprising:
a clamping part which clamps the first and second adherends in such a manner that the first and second adherends are in contact with each other;
a clamping force control unit which controls a clamping force exerted by the clamping part in such a manner that a pressure of from 9×10² to 1×10⁵ N/m² is applied to the first and second adherends clamped by the clamping part;
a heat-generating unit which heats at least one of the first or second adherends, and which comprises a resin member which generates heat upon application of vibration and a vibration part which imparts vibration to the resin member;
a heat generation control unit which, by controlling vibration of the vibration part, controls heat generation of the heat-generating unit in such a manner that the first and second adherends, being clamped by the clamping part, have a temperature of from 60 to 140° C.;
a vibration unit which vibrates at least one of the first or second adherends clamped by the clamping part; and
a vibration control unit which controls vibration generated by the vibration unit in such a manner that the first and second adherends clamped by the clamping part vibrate at a frequency of from 1 to 100 kHz.

2. The biological tissue-bonding device according to claim 1, wherein the vibration control unit controls vibration generated by the vibration unit in such a manner that the first and second adherends clamped by the clamping part vibrate at an amplitude of less than 100 μm.

3. The biological tissue-bonding device according to claim 1, wherein:
the heat-generating unit contacts one of the first or second adherends that are clamped by the clamping part and in contact with each other;
the heat-generating unit heats the adherend contacting the heat-generating unit; and
the vibration unit vibrates at least one of the first or second adherends clamped by the clamping part by vibrating at least one of the clamping part or the heat-generating unit.

4. The biological tissue-bonding device according to claim 1, wherein:
the heat-generating unit is interposed between the first and second adherends that are clamped by the clamping part and in contact with each other;
the heat-generating unit heats at least one of the first or second adherends clamped by the clamping part; and
the vibration unit vibrates at least one of the first or second adherends clamped by the clamping part by vibrating the heat-generating unit.

5. The biological tissue-bonding device according to claim 1, wherein the heat-generating unit and the heat generation control unit also serve as the vibration unit and the vibration control unit such that the heat-generating unit vibrates at least one of the first or second adherends clamped by the clamping part.

6. A biological tissue-bonding device for bonding a biological tissue which is a first adherend, and a biological tissue or a material capable of being bonded to a biological tissue which is a second adherend, the biological tissue-bonding device comprising:
a pressing part which presses one of the first or second adherends against the other;
a pressure control unit which controls pressure exerted by the pressing part in such a manner that a pressure of from 9×10² to 1×10⁵ N/m² is applied to the first and second adherends;
a heat-generating unit which heats at least one of the first or second adherends, and which comprises a resin member which generates heat upon application of vibration and a vibration part which applies vibration to the resin member;
a heat generation control unit which, by controlling vibration of the vibration part, controls heat generation of the heat-generating unit in such a manner that the first and second adherends, being pressed by the pressing part, have a temperature of from 60 to 140° C.;
a vibration unit which vibrates at least one of the first or second adherends; and
a vibration control unit which controls vibration generated by the vibration unit in such a manner that the first and second adherends vibrate at a frequency of from 1 to 100 kHz.

7. The biological tissue-bonding device according to claim 6, wherein the vibration control unit controls vibration generated by the vibration unit in such a manner that the first and second adherends vibrate at an amplitude of less than 100 μm.

8. The biological tissue-bonding device according to claim 6, wherein:
the heat-generating unit contacts one of the first or second adherends that are being pressed by the pressing part and in contact with each other;
the heat-generating unit heats the adherend contacting the heat-generating unit; and
the vibration unit vibrates the adherend contacting the heat-generating unit by vibrating the heat-generating unit.

9. The biological tissue-bonding device according to claim 6, wherein the heat-generating unit and the heat generation control unit also serve as the vibration unit and the vibration control unit such that the heat-generating unit vibrates the adherend contacting the heat-generating unit.

10. The biological tissue-bonding device according to claim 6, wherein the pressing part presses the heat-generating unit against one of the first or second adherends so as to press one of the first or second adherends against the other.

11. A heat-generating device, comprising:
a heat-generating unit having a resin member which generates heat from inside upon application of vibration, a vibration part which applies vibration to the resin member, and an adherend-contacting surface at which the resin member contacts an adherend; and
a heat generation control unit which, by controlling vibration of the vibration part, controls heat generation of the heat-generating unit in such a manner that the heat-generating unit has a prescribed temperature, wherein:
when the vibration part is vibrated while contacting the adherend-contacting surface to the adherend, the adherend is heated via the adherend-contacting surface with heat generated inside the resin member, rather than with heat generated at a portion at which the vibration part contacts the resin member and imparts vibration to the resin member.

12. The heat-generating device according to claim 11, wherein the prescribed temperature is lower than either of the melting point of the resin member or 250° C.

13. The heat-generating device according to claim 11, wherein the resin member is at least one selected from the group consisting of polytetrafluoroethylene, polyvinylidene fluoride, polyvinyl fluoride, perfluoroalkoxy fluorine resin, tetrafluoroethylene/hexafluoropropylene copolymer, ethylene/tetrafluoroethylene copolymer and ethylene/chlorotrifluoroethylene copolymer.

14. A heat-generating method, in which a resin member which generates heat from inside upon application of vibration is caused to generate heat by applying vibration to the resin member by a vibration part, wherein:
when the vibration part is vibrated while contacting an adherend-contacting surface thereof to an adherend, the adherend is heated via the adherend-contacting surface with heat generated inside the resin member, rather than with heat generated at a portion at which the vibration part contacts the resin member and imparts vibration to the resin member.

15. The heat-generating method according to claim 14, wherein the resin member is at least one selected from the group consisting of polytetrafluoroethylene, polyvinylidene fluoride, polyvinyl fluoride, perfluoroalkoxy fluorine resin, tetrafluoroethylene/hexafluoropropylene copolymer, ethylene/tetrafluoroethylene copolymer and ethylene/chlorotrifluoroethylene copolymer.
